(19) United States
(12) Patent Application Publication
(10) Pub. No.: US 2025/0318952 A1
(43) Pub. Date: Oct. 16, 2025

(54) PERINEAL WARM COMPRESS DEVICES, METHODS AND SYSTEMS

(71) Applicant: FRUITS OF LABOR INC., Weston, MA (US)

(72) Inventors: Jeong KIM, Weston, MA (US); Tesia KIM, Weston, MA (US)

(73) Assignee: FRUITS OF LABOR INC., Weston, MA (US)

(21) Appl. No.: 19/053,763

(22) Filed: Feb. 14, 2025

Related U.S. Application Data
(60) Provisional application No. 63/634,204, filed on Apr. 15, 2024.

(51) Int. Cl. A61F 7/02; A61F 7/00

(52) U.S. Cl. CPC A61F 7/02; A61F 2007/0018; A61F 2007/0288

(57) ABSTRACT

A perineal heating device includes a warming component comprising a phase change material and a disposable exterior sleeve. The perineal heating device is applied to the perineal body tissue of a patient during labor and a applies heat to maintain the perineal tissue of a patient at a desired temperature to reduce tearing and tissue trauma.

### CROSS-REFERENCE

This application claims the benefit of U.S. Provisional Application No. 63/634,204, filed Apr. 15, 2024, which application is incorporated herein in its entirety by reference.

### BACKGROUND

The Field of the Invention: The present disclosure relates generally to systems, methods, and devices for heating tissue. More specifically, the present disclosure relates to systems, methods, and devices for heating perineal body tissue during labor and delivery.

Background: According to the US Centers for Disease Control, there are approximately 2.5 million vaginal deliveries in the US annually. Worldwide, approximately 80% of births are vaginal deliveries. During a vaginal delivery, the birth canal can suffer lacerations ranging from small lacerations to severe lacerations with long term negative effects. It is estimated that more than 85% of women who give birth vaginally experience some degree of perineal trauma. Perineal trauma includes tears, grazing or episiotomy, of which about 60-70% require suturing. Large lacerations that can occur as a result of perineal trauma often require extensive repair with an increased risk of postpartum complications, including hemorrhage, infection and increased postpartum pain.

There are also long-term sequelae for mother's including incontinence of fecal material, chronic pain, increased risk of pelvic organ prolapse and the need for surgical treatment later in life. Obstetric providers and patients are interested in ways to prevent severe perineal lacerations and avoid narcotic use, which is often given for postpartum pain management.

As generally understood, perineal tears are classified into four degrees. FIG. 1A illustrates a portion of the female anatomy 10 showing the vaginal opening 12, the perineum 14, and the anus 16. with a first-degree perineal tear 30. First-degree perineal tears are the least severe and may or may not require stitches but still cause pain for the patient during and after labor and delivery. First degree perineal tears are generally limited to the tearing of the vaginal tissues and do not extend to perineal muscle tissue between the vaginal opening 12 and the anus 16.

FIG. 1B illustrates a portion of the female anatomy 10 showing a second-degree perineal tear 32. Second-degree perineal tears are more severe and involve tearing of the perineal muscles 18. These muscles support the bladder, vagina, and uterus and require stitches to repair the perineum. Second-degree perineal tears may cause the anal sphincter 20 to be exposed but does not result in a torn anal sphincter 20.

As shown in FIG. 1C, a portion of the female anatomy 10 shows a third-degree perineal tear 34 involves tearing of the perineal muscles 18 and the muscles that surround the anus 16, including the anal sphincter 20. Third-degree perineal tears also require stitches. Even after repair, long-term consequences of third-degree perineal tears can include fecal incontinence, urinary incontinence, and painful intercourse.

FIG. 1D illustrates a portion of the female anatomy 10 showing a fourth-degree perineal tear 36. Fourth-degree perineal tears are the most severe and involve complete tearing of perineal tissue and underlying muscles between the vaginal opening and the anus 16. Fourth-degree perineal tears include tearing of the perineal muscles, the anal sphincter, and the tissue lining the rectum. Significant stitching and potentially multiple surgeries may be required to repair fourth-degree tears. Long term consequences of this type of tear also include fecal incontinence, urinary incontinence, and painful intercourse.

During certain stages of labor, including later stages when cervical dilation occurs and pushing begins, including crowning of the baby's head, the perineal tissue stretches. If stretching of the perineal tissue occurs too rapidly or the stress caused by stretching is too large, the perineal tissue tears to varying degrees as described above. However, application of heat to the perineal tissue during these later stages of labor and delivery can reduce the risk of tearing of the perineal tissue.

Applying the right amount of heat to the perineal tissue at the right time requires a delicate balance. On the one hand, enough heat must be applied to the perineal tissue to reduce the risk of tearing. On the other hand, the temperature applied must not be so great that it irritates, damages, or burns the tissue. In general, the surface temperature of exposed skin in a room-temperature environment can be as low as 31° C. or 32° C. Burn temperature of the skin depends on the duration of exposure but generally start at 47° C. or higher. For example, increasing skin tissue temperature, such as perineal body tissue, to 47° C. or higher for extended periods of time, may result in burning or irritation. Temperatures greater than 47° C., such as 48° C. or higher, may be applied to skin without burning if applied for short durations. Moreover, the heat needs to be applied consistently over time in a manner that does not require excessive patient monitoring or interaction.

Current methods and devices for preventing perineal lacerations during labor and delivery include manual application of warm compresses to the perineum. Such warm compresses include, for example, a cloth soaked in warm water, which is held against the perineal tissue during labor and delivery. This results in unpredictable, cyclical temperatures being applied to the perineal tissue as the water cools during use and newly soaked cloths are reapplied. This process also requires excessive patient interaction. As will be appreciated by those skilled in the art, holding a warm compress or cloth against the perineal tissue requires one or more extra hands that may get in the way of other medical professionals performing other urgent care to the same area of the patient during labor and delivery.

Other available warm compress devices are also not conducive for use at the perineal body tissue area of a patient during labor and delivery. For example, warm compress devices often lack precise and consistent temperature control. Current heating pads and other devices are also not effectively designed for the physical contours and features of the perineal tissue and surrounding anatomy of a patient and/or for use in a wet environment such as encountered during labor and delivery. In addition, such devices may impede the use of other medical devices used during labor and delivery or block the vaginal opening or anus of the patient.

What is needed are devices, methods and systems for applying consistent heat to the perineal over an extended period of time which does not require excessive patient interaction by a healthcare provider.

### SUMMARY

Disclosed are devices, methods and systems for applying consistent heat to the perineal over an extended period of time which does not require excessive patient interaction by a healthcare provider.

Suitable devices include a perineal warming device comprising: a flexible warming component comprising a phase change material operable to maintain a therapeutic temperature for about 2 to 10 hours wherein the flexible warming component has an upper surface with a plurality of upper surface recesses; a flexible warming component sleeve operable to encapsulate the flexible warming component; and a disposable exterior sleeve with an interior cavity.

Additional devices include a perineal warming device comprising: a flexible warming component comprising a phase change material operable to maintain a therapeutic temperature of from 105° F. to 115° F. (40.6° C. to 46.1° C.), for a therapeutic amount of time wherein the flexible warming component has an upper surface with a plurality of upper surface recesses; a flexible warming component sleeve operable to encapsulate the flexible warming component; and a disposable exterior sleeve with an interior cavity.

Systems are provided for perineal warming which include, for example, perineal warming devices, and replaceable warming device sleeves.

Methods of using the disclosed perineal warming devices comprise: obtaining a flexible warming component; warming the flexible warming component to a target temperature; and placing the flexible warming component adjacent a perineal tissue of a patient.

Kits are also provided which include, for example, perineal warming devices, and replaceable warming device sleeves.

Both the foregoing general description and the following detailed description are exemplary and explanatory only and are not restrictive of the disclosed embodiments, as claimed.

### INCORPORATION BY REFERENCE

All publications, patents, and patent applications mentioned in this specification are herein incorporated by reference to the same extent as if each individual publication, patent, or patent application was specifically and individually indicated to be incorporated by reference.
CN213076181U published Apr. 30, 2021 to Du Jisha;
GB2584068A published Nov. 25, 2020 to Keith;
KR200281049Y published Jul. 12, 2002 to Park;
US2012/0109232A1 published May 3, 2012 to Mohn;
US2015/0366367A1 published Dec. 24, 2015 to Augustine et al.;
US2017/0135855A1 published May 18, 2017 to Stefan et al.;
US2021/0038259A1 published Feb. 11, 2021 to Langell et al.;
US2024/0189141A1 published Jun. 13, 2024 to Augustine et al.;
U.S. Pat. No. 6,329,644B1 issued Dec. 11, 2001 to Hyatt;
U.S. Pat. No. 9,314,400B1 issued Apr. 19, 2016 to Dudley;
WO2019/132775A1 published Jul. 4, 2019 to Leow et al.; and
Moist Heating Pad with Auto Shut Off for Cramps and Back Pain by ThermoRelief (2024) (available on amazon.com).

### BRIEF DESCRIPTION OF THE DRAWINGS

The novel features of the invention are set forth with particularity in the present disclose. As will be appreciated by those skilled in the art, the various features illustrated in the drawings may not be drawn to scale. Moreover, the illustrations presented in the present disclosure are not meant to be actual views of any particular apparatus (e.g., device, system, etc.) or method, but are merely idealized representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or all operations of a particular method.

A better understanding of the features and advantages of the present invention will be obtained by reference to the following detailed description that sets forth illustrative embodiments, in which the principles of the invention are utilized, and the accompanying drawings of which:

FIG. 1A illustrates a portion of the anatomy with a first-degree perineal laceration;

FIG. 1B illustrates a portion of the anatomy with a second-degree perineal laceration;

FIG. 1C illustrates a portion of the anatomy with a third-degree perineal laceration;

FIG. 1D illustrates a portion of the anatomy with a fourth-degree perineal laceration;

FIG. 2 is an exploded view of a perineal warming device;

FIGS. 3A-B are an image of an inner warming core of a perineal warming device, and a disposable outer device covering;

FIGS. 4A-E are images of an exterior surface of inner warming core of a perineal warming device;

FIG. 5 is a warming device suitable to heat the perineal warming devices prior to use;

FIGS. 6A-D illustrate steps for deploying a perineal warming device; and

FIG. 7 illustrates a perineal warming device applied to an interior of a patient during use.

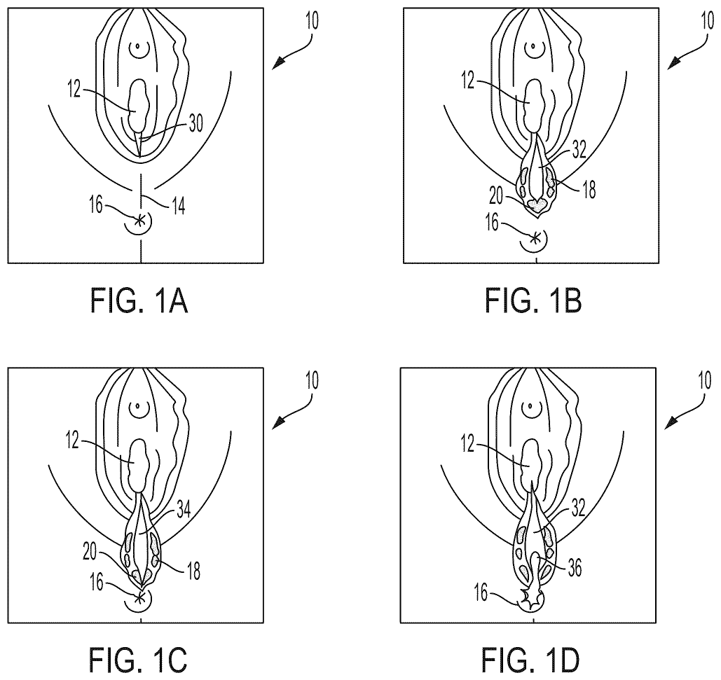

### DETAILED DESCRIPTION

### I. Devices

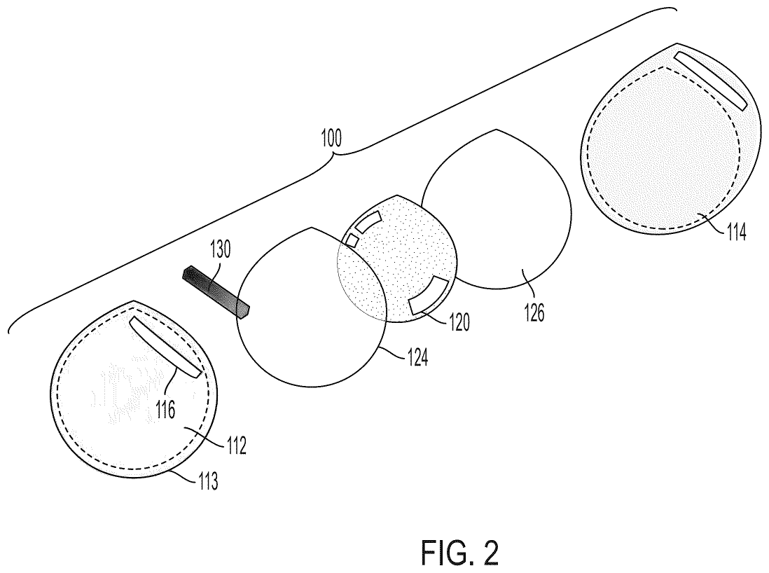

Turning now to FIG. 2 an exploded view of a perineal warming device 100 is illustrated. The shape of the perineal warming device 100 and its components can be round, oval, teardrop, or any other suitable shape. The size of the perineal warming device 100 ranges from about 8 cm to 15 cm in diameter, more preferably 10 cm and from about 2 cm to 6 cm in thickness. The perineal warming device 100 is formed from a soft, flexible material that is conformable to the perineal area of a patient during use. The perineal warming device 100 has an upper surface with an upper surface area, a lower surface with a lower surface area and a thickness. The perineal warming device 100 is operable to generate a therapeutic level of warmth for an extended period of time during labor. Suitable therapeutic temperatures maintainable by the perineal warming device 100 include therapeutic temperature ranges from 105° F. to 115° F. (40.6° C. to 46.1° C.), more preferably 110° F. (43.3° C.). The perineal warming device 100 is operable to achieve and maintain the therapeutic temperature for a therapeutic period of time of, for example, from 1 hour to 8 hours, more preferably 2-6 hours, even more preferably 4 hours. The perineal warming device 100 further comprises two components, a warming component 120 and a disposable exterior sleeve 110.

The disposable exterior sleeve 110 having has a first disposable exterior sleeve surface 112, a second disposable exterior sleeve surface 114, and an exterior sleeve opening 116. The first disposable exterior sleeve surface 112 and second disposable exterior sleeve surface 114 defines an exterior sleeve cavity operable to receive a warming component 120 therein when the first disposable exterior sleeve surface 112 is secured to the second disposable exterior sleeve surface 114 about an exterior edge 113 of the first disposable exterior sleeve surface 112 and the second disposable exterior sleeve surface 114. The flexible warming component sleeve can be formed from, for example, low-density polyethylene (LDPE).

The warming component 120 has a phase change core 122. The phase change core 122 can further be encapsulated by a flexible warming component sleeve 124 having a first flexible warming component sleeve surface and a second flexible warming component sleeve surface. The flexible warming component sleeve 124 can be formed from a suitable plastic such as low-density polyethylene (LDPE). LDPE provides a flexible, lightweight and transparent plastic that is non-toxic and non-contaminating and separates the phase change core 122 from the patient during use. The phase change core 122 can be formed from suitable phase change materials including, for example, phase change wax, medical-grade silicone, viscoelastic polymer, latex-free elastomers, paraffin wax, n-elicosane, n-hemeicosane, n-docosane, n-tricosane, n-tetracosane, n-pentacosane, n-hexacosane, n-heptacosane, n-Octacosane, and combinations thereof. As will be appreciated by those skilled in the art, other suitable phase change materials can be used without departing from the scope of the disclosure. The phase change material forming the phase change core 122 is operable to achieve and maintain a therapeutic temperature for an extended period of time of, for example, 2-4 hours.

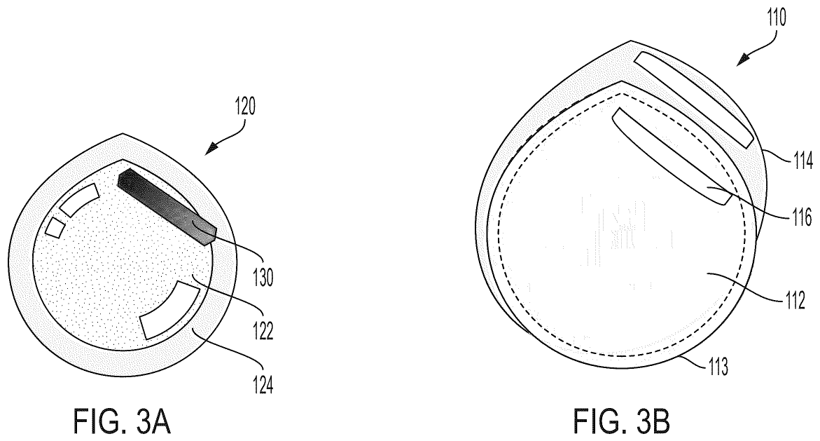

FIGS. 3A-B are an image of a phase change core 122 of a perineal warming device 100, and a flexible warming component sleeve 124 (FIG. 3A) and a disposable exterior sleeve 110 (FIG. 3B). A safety indicator 130 is provided. The safety indicator 130 is operable to provide temperature information to the user. The safety indicator 130 can be incorporated into the flexible warming component sleeve 124 (as shown), or incorporated into the phase change core 122, or the disposable exterior sleeve 110 without departing from the scope of the disclosure. The temperature information provided by the safety indicator 130 enables the user to know whether the perineal warming device 100 is too cool (therefore ineffective), too warm (therefore potentially damaging or irritating), or just right (therefore therapeutically effective) at or during deployment of the warming device. The safety indicator 130 provides the user with a visual indication to determine if the perineal warming device is too hot or has cooled to a point where it is no longer efficaceous. An example of a safety indicator includes a thermochromic strip using a thermochromic ink or coating, such as the thermochromic ink available from LCR Hallcrest (https://www.thermographics.com/). Suitable thermographic ink should include the an ability to indicate when the device is at least within the therapeutic temperature range. More preferably, the thermochromic ink is operable to indicate when for example, the perineal warming device is heated to, for example, 150° F. Although the thermographic ink can indicate when the perineal warming device is too cold (e.g., below 105° F. (40.6° C.), changes to the phase change material may also be used as an indicator of cooling. The flexible warming component sleeve 124 can be formed from, for example, cotton, polyethylene, polypropylene, polyvinyl chloride, ethylene vinyl acetate, or rayon. Other materials, including thermochromic pigments and liquid crystals encapsulated in a plastic film, can be used without departing from the scope of the disclosure. The safety indicator can be adhered to either the phase change core or the warming component sleeve, for example, using a suitable biocompatible adhesive.

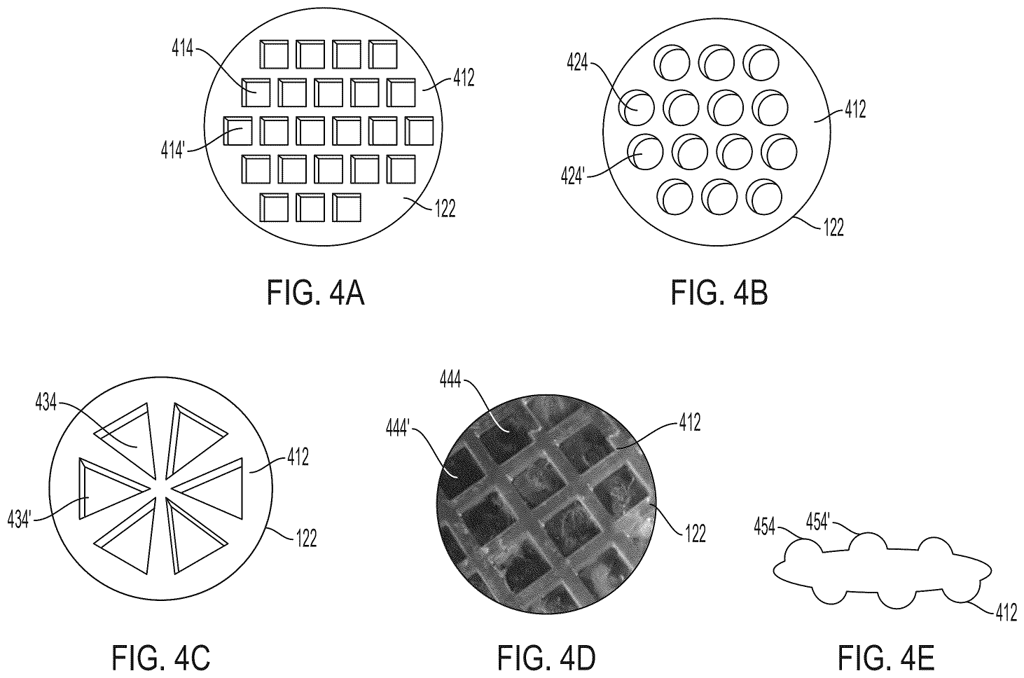

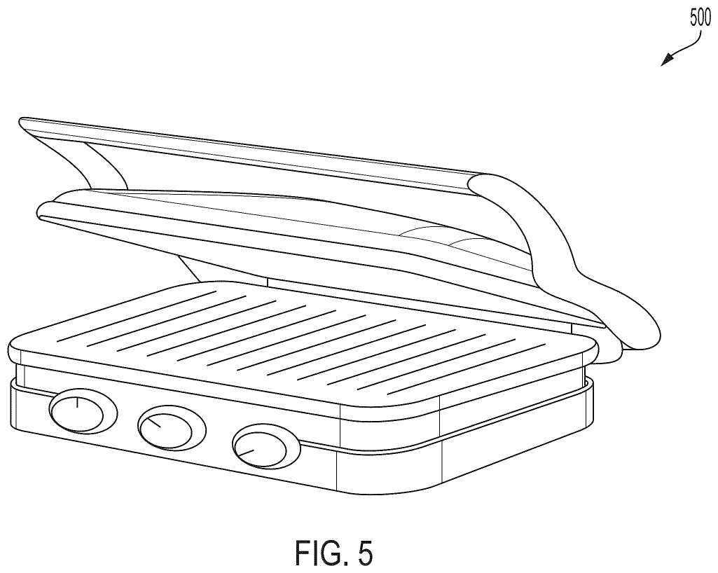

FIGS. 4A-E are images of an exterior surface of phase change core 122 of a perineal warming device, wherein at least one exterior surface is not planar. The non-planar surface has a plurality of recesses, indentations or set-backs operable to reduce the effective surface area of the upper most patient skin engaging surface. The non-planar surface can also have convex or concave surface treatment. As shown in FIG. 4A the upper surface 412 of the phase change core 122 has a plurality of recesses 414, 414′ that have a square shape. The plurality of recesses 414, 414′ reduce the effective surface area of the upper surface 412 of the phase change core 122. Reducing the effective surface area of the upper surface 412 reduces the surface area of the inner warming phase change core 122 that comes into contact with the perineal surface of the patient and is operable to increase the length of time the inner warming phase change core 122 is able to maintain the therapeutic temperature during use. The phase change core 122 can have a plurality of upper surface recesses and a plurality of lower surface recesses.

For a perineal warming device formed from phase change material that contains water with a 10 cm upper surface area that is 2 cm thick and heated to a starting temperature of 110° F., it would take about 8 hours to cool to a room temperature (e.g., a bout 70° F. to 75° F. (21° C. to 24° C.)). If the thickness of the perineal warming device is increased to 3 cm, it takes approximately 10 hours to cool to room temperature.

For a perineal warming device with a 10 cm upper surface area that is 2 cm thick formed from phase change wax, it would take about 4 hours to cool to a sub-therapeutic temperature (e.g., <105° F. (40.6° C.)). Where the 10 cm upper surface has a plurality of recesses resulting in an effective surface area of the upper surface of 3 cm the effective warming time could be extended to about 6 hours.

FIG. 4B illustrates the upper surface 412 of the inner warming core 120 has a plurality of recesses 424, 424′ that have a round shape. FIG. 4C illustrates the upper surface 412 of the inner warming core 120 has a plurality of recesses 434, 434′ that have a triangular shape. Other shapes and orientations can be used without departing from the scope of the disclosure. FIG. 4D illustrates the upper surface 412 of the inner warming core 120 has a plurality of recesses 444, 444′ forming a grid pattern. In yet another configuration, the upper surface 412 of the inner warming core 120 has a plurality of convex members 454, 454′ extending therefrom. Additionally, both the upper exterior surface of the inner warming core and the opposing lower exterior surface of the inner warming core can have a plurality of recesses and/or convex or concave members or features operable to change the overall tissue engaging surface area of the warming device without departing from the scope of the disclosure.

### II. Methods

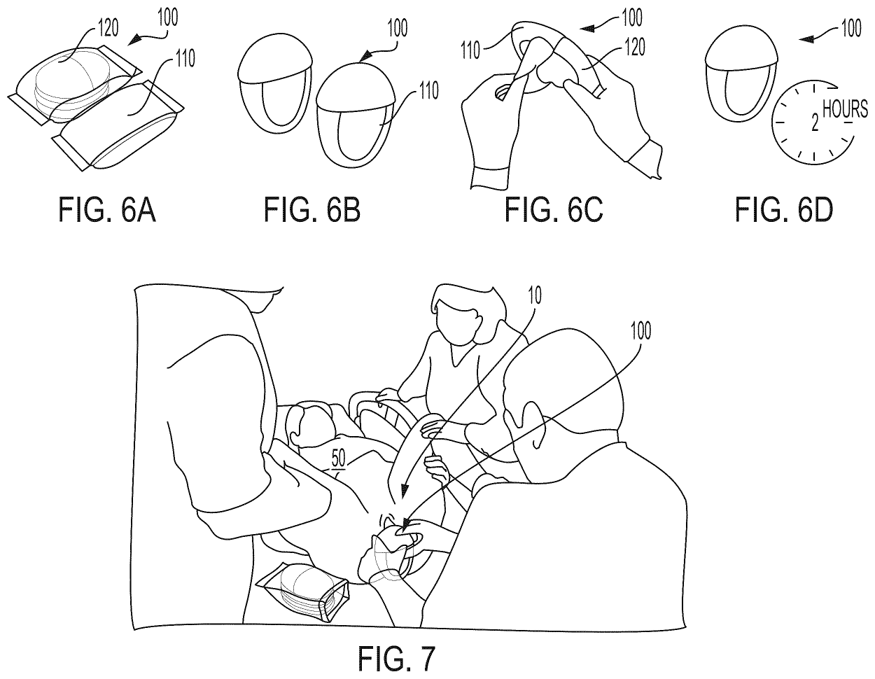

FIGS. 6A-D illustrate steps for deploying a perineal warming device 100.

Initially, the warming component 120 and the disposable exterior sleeve 110 are provided in separate packaging as shown in FIG. 6A. The warming component 120 is stored in a hospital warmer (e.g., hospital warmer 500 in FIG. 5) to allow the warming component to be deployed at or near a target temperature. The warming component 120 and the disposable exterior sleeve 110 are removed from their packaging as shown in FIG. 6B. The user determines if the temperature of the warming component 120 is optimal for deployment by looking at the safety indicator. The user then inserts the warming component 120, which has been warmed to a therapeutic temperature, into the interior of the disposable exterior sleeve 110 as shown in FIG. 6C. The perineal warming device 100 is then ready to deploy by placing the warming device on the patient's perineal tissues for a period of time as shown in FIG. 6D. When the disposable exterior sleeve 110 becomes soiled, the disposable exterior sleeve 110 can be removed and replaced.

FIG. 7 illustrates a perineal warming device 100 applied to a portion of the female anatomy 10 of a patient 50 during use.

### Iii. Kits

Kits comprise at least one perineal warming device 100 having a warming component 120 and a disposable exterior sleeve 110. Additional disposable sleeves can be provided as part of the kit. Moreover, the warming component can be provided in a sealed warming component package, and one or more exterior sleeves can be provide in a sealed exterior sleeve package that is separate from the warming component package. Instructions for use and warnings can also be provided.

As will be appreciated by those skilled in the art, throughout this specification, unless the context requires otherwise, the word “comprise” or variations such as “comprises” or “comprising”, will be understood to imply the inclusion of a stated integer or group of integers but not the exclusion of any other integer or group of integers.

While preferred embodiments of the present invention have been shown and described herein, it will be obvious to those skilled in the art that such embodiments are provided by way of example only. Numerous variations, changes, and substitutions will now occur to those skilled in the art without departing from the scope of the invention. For example, the use of comprise, or variants such as comprises or comprising, includes a stated integer or group of integers but not the exclusion of any other integer or group of integers. It should be understood that various alternatives to the embodiments of the invention described herein may be employed in practicing the invention. It is intended that any claims presented at any time in this application define the scope of the invention and that methods and structures within the scope of these claims and their equivalents are covered thereby.

What is claimed:

1. A perineal warming device comprising:
a flexible warming component comprising a phase change material operable to maintain a therapeutic temperature for about 2 hours wherein the flexible warming component has an upper surface with a plurality of upper surface recesses;
a flexible warming component sleeve operable to encapsulate the flexible warming component; and
a disposable exterior sleeve with an interior cavity.

2. The perineal warming device of claim 1 wherein the plurality of upper surface recesses have a shape selected from round, square, rectangular, triangular, teardrop, oval, and ovoid and are convex or concave.

3. The perineal warming device of claim 1 wherein the upper surface of the flexible warming component has a first upper surface area determined by a shape of the flexible warming component and a second upper surface area, less than the first upper surface area, determined by a shape of the upper surface recesses.

4. The perineal warming device of claim 1 wherein a lower surface of the flexible warming component has a plurality of lower surface recesses with a shape selected from round, square, rectangular, triangular, teardrop, oval, and ovoid.

5. The perineal warming device of claim 4 wherein the lower surface of the flexible warming component has a first lower surface area determined by a shape of the flexible warming component and a second lower surface area, less than the first lower surface area, determined by a shape of the plurality of lower surface recesses.

6. The perineal warming device of claim 1 wherein the flexible warming component sleeve is formed from low-density polyethylene (LDPE).

7. The perineal warming device of claim 1 wherein the disposable exterior sleeve is formed from one or more of cotton, polyethylene, polypropylene, polyvinyl chloride, ethylene vinyl acetate, rayon.

8. The perineal warming device of claim 1 wherein the therapeutic temperature ranges from 105° F. to 115° F. (40.6° C. to 46.1° C.).

9. A perineal warming device comprising:
a flexible warming component comprising a phase change material operable to maintain a therapeutic temperature of from 105° F. to 115° F. (40.6° C. to 46.1° C.), for a therapeutic time wherein the flexible warming component has an upper surface with a plurality of upper surface recesses;
a flexible warming component sleeve operable to encapsulate the flexible warming component; and
a disposable exterior sleeve with an interior cavity.

10. The perineal warming device of claim 9 wherein the plurality of upper surface recesses have a shape selected from round, square, rectangular, triangular, teardrop, oval, and ovoid and are convex or concave.

11. The perineal warming device of claim 9 wherein the upper surface of the flexible warming component has a first upper surface area determined by a shape of the flexible warming component and a second upper surface area, less than the first upper surface area, determined by a shape of the upper surface recesses.

12. The perineal warming device of claim 9 wherein a lower surface of the flexible warming component has a plurality of lower surface recesses with a shape selected from round, square, rectangular, triangular, teardrop, oval, and ovoid.

13. The perineal warming device of claim 12 wherein the lower surface of the flexible warming component has a first lower surface area determined by a shape of the flexible warming component and a second lower surface area, less than the first lower surface area, determined by a shape of the plurality of lower surface recesses.

14. The perineal warming device of claim 9 wherein the flexible warming component sleeve is formed from low-density polyethylene (LDPE).

15. The perineal warming device of claim 9 wherein the disposable exterior sleeve is formed from one or more of cotton, polyethylene, polypropylene, polyvinyl chloride, ethylene vinyl acetate, rayon.

16. The perineal warming device of claim 9 wherein the therapeutic time ranges from at least 1 hour to 8 hours.

17. A method of using a perineal warming device comprising:
obtaining a flexible warming component comprising a phase change material wherein the flexible warming component has an upper surface with a plurality of upper surface convex or concave recesses, a flexible warming component sleeve operable to encapsulate the flexible warming component;
warming the flexible warming component to a target temperature; and
placing the flexible warming component adjacent a perineal tissue of a patient.

18. The method of using a perineal warming device of claim 17 further comprising: placing the flexible warming component in an interior cavity of a disposable exterior sleeve.

19. The method of using a perineal warming device of claim 18 further comprising: replacing the disposable exterior sleeve with a new disposable exterior sleeve.

20. A kit comprising:
a flexible warming component comprising a phase change material wherein the flexible warming component has an upper surface with a plurality of upper surface recesses, a flexible warming component sleeve operable to encapsulate the flexible warming component; and
one or more disposable sleeves.
